(19) United States
(12) Patent Application Publication
(10) Pub. No.: US 2003/0033016 A1
(43) Pub. Date: Feb. 13, 2003

(54) Implant sheath

(75) Inventor: Roger Ryan Dees JR., Atoka, TN (US)

Correspondence Address: OLIFF & BERRIDGE, PLC, P.O. BOX 19928, ALEXANDRIA, VA, 22320, US

(73) Assignee: GYRUS GROUP PLC, Wokingham (GB)

(21) Appl. No.: 09/922,860

(22) Filed: Aug. 7, 2001

(51) Int. Cl. A61F 2/18

(52) U.S. Cl. 623/10; 206/438

(57) ABSTRACT

A sheath for protection and stabilization of a medical implant during shipping and manipulation. The sheath protects a small or fragile medical implant from damage, breakage or loss during shipping, and provides support for trimming and other handling by a surgeon or other medical staff member. The sheath is made from a trimmable material and has at least one flat side to assist in manipulation of the medical implant.

### FIELD OF THE INVENTION

This invention relates generally to devices for protection and stabilization of a medical implant during shipping and manipulation, and more particularly to protection, stabilization and support of small and fragile medical implants, such as otologic implants, while maintaining sterility.

### BACKGROUND OF THE INVENTION

Various packages for delivery of medical implants have been created in recent years in an effort to provide enhanced protection of small medical implants during shipping and to facilitate removal of the small implant from the sterile delivery package. The inventors recognize these problems and the need for a packaging system which would provide improved protection and support for the medical implant during both shipping and manipulation.

Medical implants come in numerous shapes and sizes and are either very small, very fragile, or both. These properties make shipping, storage and manipulation of these implants difficult. Due to the numerous shapes and sizes of the implants and the costs associated with tooling new package designs, custom packaging of small medical implants has not been feasible. Medical implants, such as otologic implants, require packaging for shipping that protects the implants and retains the sterile properties of the implants. Otologic implants replace the small bones of the middle ear, the malleus, the incus and the stapes. The small bones of the middle ear receive vibrations of sound from the tympanic membrane.

Generic packaging has been utilized to package most Ear Nose and Throat implants. This generic packaging has one or more cavities and a lid for closing the cavities, permitting the smaller implants to move about inside the package resulting in cracks, breaks or other deformities of the implants. Unfortunately, surgeons often find small medical implants broken in numerous pieces upon opening the generic packaging during the surgical procedure.

Additionally, manipulation and modification of these implants is normally required upon receipt by the surgeon; however, the fragile nature of these implants makes proper handling and modification difficult. Thus, breakage of the implants is a frequent problem during both shipping and manipulation.

The implant may be modified by cutting or trimming in order to obtain a correct fit for each individual patient. The fragile nature of otologic implants makes manipulation of the implants on a flat surface using a cutting instrument, such as a scalpel, a difficult maneuver.

An otologic implant generally has a head portion, a neck portion, and a shaft portion. Breakage of otologic implant usually occurs at the neck, which is the weakest point of the implant, located just below the head. An otologic implant may also be damaged due to compression of the shaft during trimming or cutting. The diameter of the head is often larger than the shaft, which creates an angle of the implant's centerline so that only points near each end of the implant are properly supported during trimming, inducing stress at the weakest points. Stress cracks, breaks, compression and other deformities may result from pressure exerted on the implant from the cutting instrument. Thus, it is difficult to obtain the desired length while avoiding damage to the implant. Surgeons often return damaged otological implants to their manufacturer. Upon return the implants evidence the fact that an attempt to trim the shaft was made by a smooth cut into less than ¼ of the shaft followed by a jagged fracture. Also, the implants are often returned broken at the intersection of the head and the shaft.

What is needed is a packaging system that would stabilize and protect small medical implants during shipment and manipulation.

### SUMMARY OF THE INVENTION

Methods and devices according to this invention seek to provide a medical implant sheath which includes an outer surface having at least one flat side and a bore that is adapted to receive and retain a shaft of a medical implant. The bore secures the shaft of the medical implant sufficiently snugly to allow the surgeon to accurately trim the shaft of the implant to a desired length by cutting through the sheath and the implant simultaneously. Accordingly, reference marks can be placed on the medical implant sheath to assist the surgeon in accurately cutting the shaft of the medical implant to a desired length.

A sheath according to the present invention, unlike current medical implant packages, is made of a material that has properties which promote support yet is able to be trimmed by a cutting instrument such as a scalpel. This feature provides a medical implant sheath that can function as a protective sleeve and stabilizer during shipment as well as during manipulation by the surgeon. The three-dimensional support provided by the medical implant sheath reduces the need for replacement implants due to problems involved in trimming, damage during shipment or misplacement of the implant upon opening a standard shipping package. The additional support also reduces the deformities of the shaft portion of the medical implant often associated with post operative complications.

Reference marks on the sheath aid the surgeon in handling and manipulation of the medical implant. Since medical implants come in standard sizes, the shaft of the medical implant must be trimmed to a desired length for each patient. Conventional shipping packages require the surgeon to remove the medical implant from the shipping package and then cut the shaft to the desired length, which creates undue strain on medical implant. Removing the implant from the shipping package for trimming, as required by conventional shipping packages, also decreases the accuracy in trimming the shaft of an implant to the desired length. The bore of the sheath also aids the surgeon in accurately trimming the shaft of the medical implant to a desired length by retaining the shaft, allowing the surgeon to simultaneously cut through the sheath, at the reference mark, and the shaft of the medical implant

One feature of a medical implant sheath according to this invention is the ability to retain the implant securely in a shipping package during shipment. Another feature of a sheath according to this invention is the prevention of breakage and deformities of medical implants during shipment and modification by providing stabilization and support. Yet another feature of a sheath are reference marks for accomplishing truer perpendicular cuts of the implant.

These and other features of this invention will become apparent after a review of the following detailed description of the disclosed embodiments.

### BRIEF DESCRIPTION OF THE DRAWINGS

FIG. 1 is a perspective view of a medical implant sheath and an implant according to one embodiment of this invention.

FIG. 2 is a perspective view of the cutting sheath of FIG. 1, together with an otologic implant, as packaged.

FIG. 3 is a perspective view of the sheath and implant of FIG. 2, in packaging suitable for shipping.

FIG. 4 is a perspective view of the sheath and implant of FIG. 2, showing the sheath and implant after trimming.

FIG. 5 is a side elevational view of the sheath and implant of FIG. 2.

FIG. 6 is a cross-sectional view of the sheath and implant of FIG. 2.

FIG. 7 shows an otologic implant that is broken at the neck.

FIG. 8 shows an otologic implant that is compressed at the cut of the shaft.

FIG. 9 shows an otologic implant that has an improper non-perpendicular cut.

### DETAILED DESCRIPTION

Methods and devices according to this invention seek to provide stabilization and protection of a medical implant during shipment and support of the implant during modifications performed by the surgeon. A sheath according to this invention houses a small or fragile medical implant, providing needed protection and support and reducing stress on the implant. In one embodiment, a sheath according to this invention houses an otological implant.

Implants used with a sheath according to this invention preferably have a length from about 3.0 mm to about 8.0 mm and a width of about 1.0 mm; some features of implants used with the sheath of the present invention are as small as 0.5 mm. Implants used in accordance with the present invention are preferably made from hydroxyapatite, which is a powdered material, but when fired is similar to ceramic. Implants and devices made from hydroxyapatite, including ear canal walls and implants for vocal cords, are machined rather than molded. Machining eliminates the surface tensions and micro-structures familiar in molded processes. Machining the implants makes them less susceptible to cracks and breaks. Hydroxyapatite is very hard and brittle and is usually trimmed by utilizing a diamond burr. Placing the hydroxyapatite implant in a sheath adds support, thereby reducing breakage. A sheath according to this invention is useful for any otological or small implant made by any process with any material.

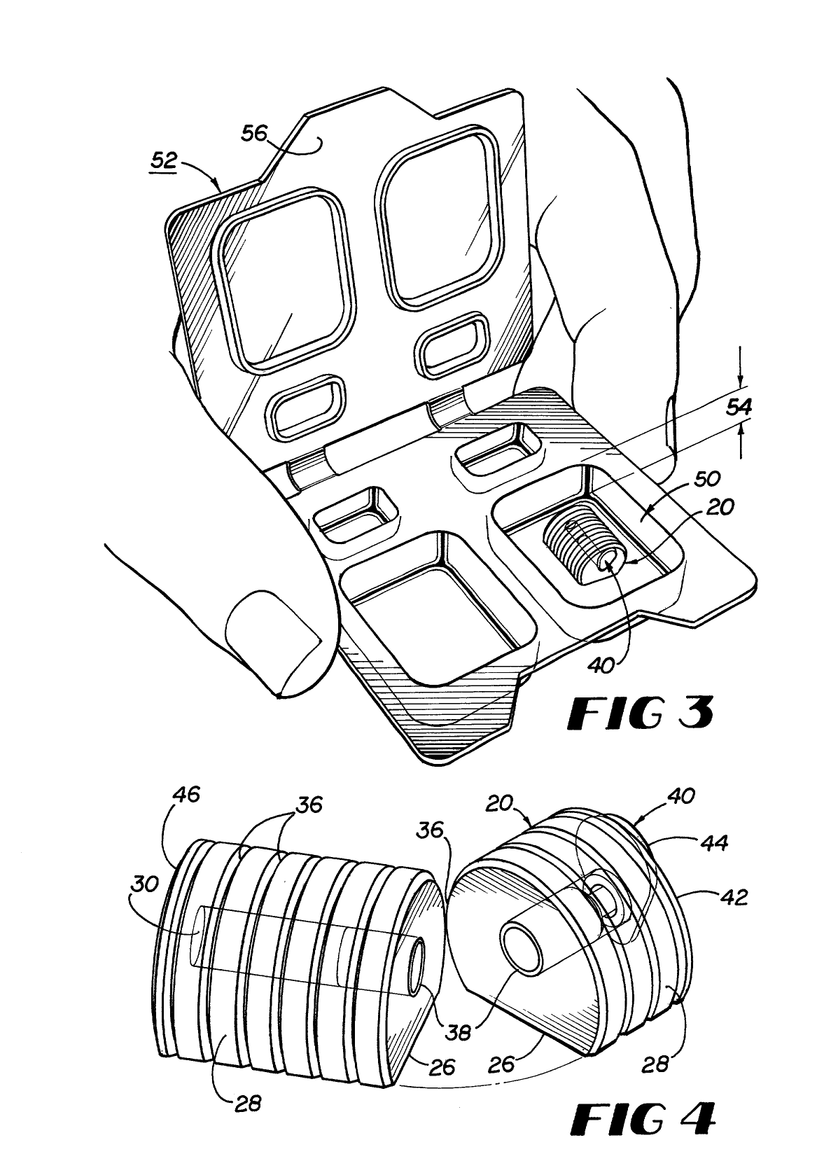

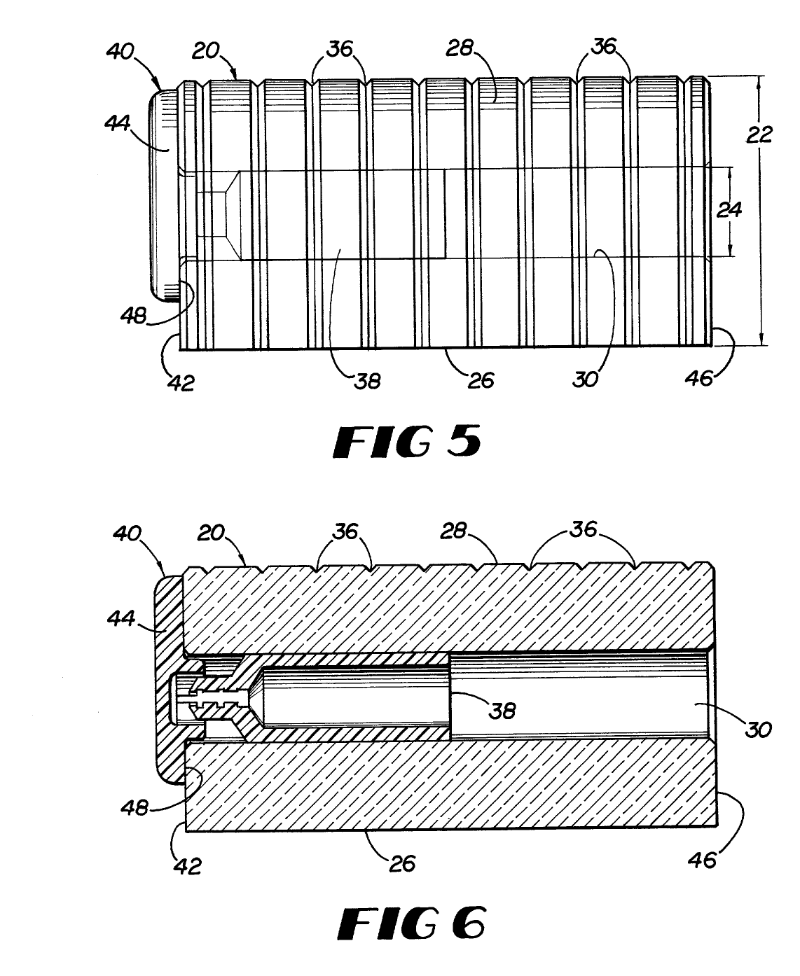

Various embodiments of the present invention are shown in FIGS. 1-6. Referring first to FIG. 1, sheath 20 has height 22 and inner diameter 24. Preferably, sheath 20 has a flat surface 26 which prevents movement of sheath 20, thereby allowing sheath 20, containing the implant, to remain stable during shipment and manipulation. Preferably, outer surface 28 is rounded; however, outer surface 28 may be square, rectangular, triangular, or any other suitable shape.

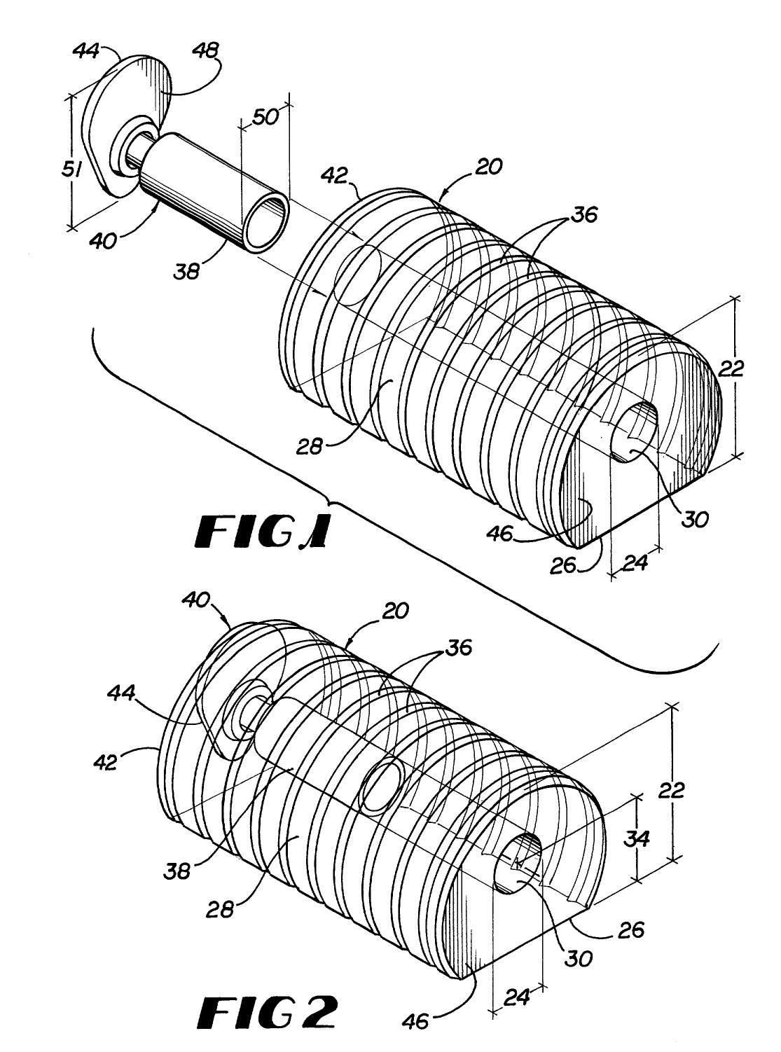

As shown in FIG. 2, bore 30 is adapted to receive and retain an implant. According to one embodiment of the present invention, bore 30 may extend the entire length of sheath 20, thereby allowing medical implants of various lengths to be shipped in a standard sheath. Height 22 of sheath 20 may correspond to the height of the shipping packaging. Preferably, height 22 of sheath 20 slightly exceeds the height of the package, such as a blue mold, so that the sheath is slightly compressed in the packaging, thereby securely retaining the sheath and the implant in place during shipment. For example, as shown in FIG. 3, if a standard blue mold has an inner height of 6 mm, a sheath that accompanies that blue mold may have a height 22 that is slightly greater than about 6.5 mm.

Referring now to FIG. 3, the implant 40 and sheath 20 are packaged for shipment in a cavity 50 within the blue mold 52. The cavity 50 had a height 54 that is slightly less than the height 22 of the sheath. The blue mold is a clam shell arrangement constructed of a resilient material with a snap lid 56 whereby when the lid is closed, the implant retaining the sheath are between the interior surfaces of the lid 56 and the cavity 50 to prevent movement.

Center 34 of sheath 20 may have a height equal to about one-half the total width 51 of the implant. Preferably, bore 30 is centered in sheath 20 so that the outer edges of the sheath 20 extend beyond the head of the implant. In this embodiment, the head of the implant is prevented from contacting the packaging, thereby protecting the head of the implant in the shipping package.

Sheath 20 may be made from silicon, fluroplast or other suitable material. If fluroplast is the material used, sheath 20 may be machined. The fluroplast material has similar flexibility as the implant. If sheath 20 is made from silicon, sheath 20 may be manufactured in an injection molding process. Inner diameter 24 varies depending upon the diameter of the shaft of the implant to be used. The same injection mold may be used to make each size, with the internal diameters varying depending on the pin used. The injection molding process forces the silicon material through a heating element and into a tool containing the sheath design. As the silicon cools, it retains the shape of the tool. The silicon material is very elastic, thereby making it more flexible than the implant it is being used to protect. A sheath according to this invention may be made from any desired material according to any desired process.

As shown in FIGS. 1 and 2, incremental reference marks 36 may be placed in certain increments along sheath 20. In one embodiment, shown in FIGS. 1-5, reference marks 36 are grooves that may be spaced in 0.5 mm increments. Alternatively, reference marks 36 are grooves placed at 1.0 mm increments. More preferably, one end of sheath 20 contains reference marks spaced at 0.5 mm increments, while the second end of sheath 20 contains reference marks spaced at 1.0 mm increments. Preferably, reference marks 36 may be grooves, as shown in FIGS. 1-5, or they may be made with ink, laser or chemical etching, or made with any other similarly suitable method of marking. The starting point for the reference marks may vary depending upon the functional dimensions of the implant.

In one embodiment, sheath 20 may be used with an implant 40, such as an incus prosthesis, as shown in FIGS. 2-6. Shaft 38 of implant 40 is received in a first end 42 of sheath 20. Head 44 of implant 40 protrudes from first end 42 of sheath 20. In one embodiment, inner side 48 of head 40 is adjacent to first end 42 of sheath 20 and shaft 38 of implant 40 does not extend beyond second end 46 of sheath 20. In another embodiment. Inner side 48 of head 50 is adjacent to first end 42 of sheath 20 and the shaft 38 of the implant extends beyond second end 46 of sheath 20. Preferably, inner diameter 24 of sheath 20 at least approximately matches the diameter 50 of shaft 38, thereby preventing gravity from dislodging implant 40 from sheath 20. In this manner, implant 40 is securely retained in sheath 20.

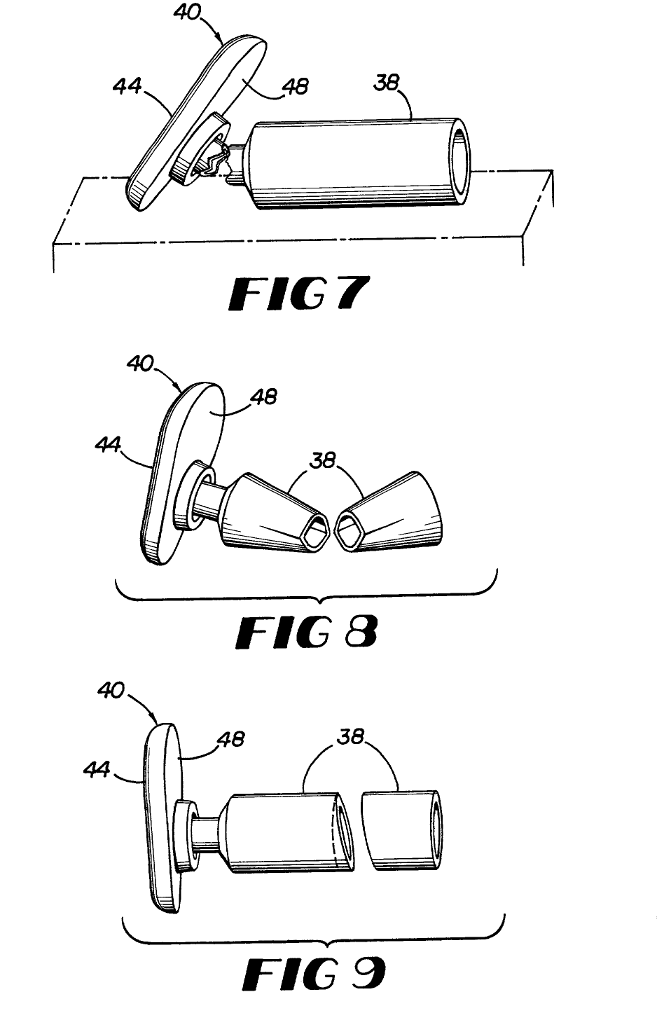

Modifications of shaft 38 of implant 40 may be performed while implant 40 is securely retained in sheath 20. In one embodiment, a surgeon may place sheath 20 retaining implant 40 with flat surface 26 on a table or other suitable cutting surface. Trimming or cutting may be accomplished without requiring contact of head 44 of implant 40 with the cutting surface. In this manner, sheath 20 provides a parallel part centerline and eliminates undue strain on implant 40, thus reducing breakage, as shown in FIG. 7, compression, as shown in FIG. 8, and other deformities. Shaft 38 is held at the correct angle in sheath 20, reducing the incidence of an incorrect angle cut, as often results without use of sheath 20, and as is shown in FIG. 9.

In an alternative embodiment, a sheath according to this invention is used with a piston or stapes prosthesis, which is an otologic implant that fits around the incus in the event the connection bone is disjointed from the footplate. A piston prosthesis includes a hook made out of wire, which bends very easily. The sheath of this embodiment protects the shaft of the piston, allowing the wire hook to extend beyond the end of the sheath. In one embodiment, the sheath height is greater than the height of the wire hook, so that the hook is protected during cutting or trimming. Other alternative embodiments include a sheath for use with various other medical implants, including other Stapes prostheses, PORP® prostheses, and TORP® prostheses.

Prior to being placed into the sheath, the implant is inspected and cleaned. Since the implant is extremely delicate, it must be handled with great care. The implant is handled with tweezers and gently pushed into the sheath. Once the implant is placed in the sheath, the sheath may be placed into a generic packaging. The generic packaging containing the sheath can be labeled and placed in a sealed tray made of a vacuum-formed plastic bottom and a TYVEK® shield. The sealed tray can then be placed into a cardboard box and shrink-wrapped with plastic. The sealed tray, containing the generic packaging with the sheath and implant, can then be sterilized in approved sterilization methods, such as Ethylene-Oxide.

Prior to trimming, the sterilized tray containing the generic packaging with the sheath and implant is placed on a tray by a nurse. The surgeon measures the patient's ear to determine the length of the implant needed. The surgeon can then place the sheath containing the implant on a sterile surface and using a scalpel, or other suitable cutting instrument, simultaneously cut the sheath and implant to the desired length. The implant is then removed from the sheath using tweezers and washed with saline.

Generally, if the implant retained in the sheath is an incus prosthesis, the nurse places the implant on a curved needle with the notch side of the implant up, which allows the surgeon to place the tip of the needle on the stapes capitulum. The implant is then slid off the needle directly onto the stapes using a 20-guage suction tip. A 2 mm right-angled hook is then used to lift the malleus while the implant is advanced beneath it. The hook portion of the prosthesis may be angled inferiorly or superiorly along the malleus handle to increase the stability of the implant. A slight angulation of the prosthesis may exist as it rests between the malleus handle and the surface of the tympanic membrane.

Generally, if the implant retained in the sheath is an incus-stapes prosthesis, the implant is passed into the middle ear by placing a 20-gauge suction tip onto the body of the implant while the surgeon's thumb covers the control hole. The force of the vacuum holds the implant in place while a 1 mm right-angled hook assists in positioning the implant over the stapes footplate and just posterior to the malleus. A 2 mm right-angled hook is then used to lift the malleus while the implant is advanced beneath it.

Generally, if the implant contained in the sheath is a malleus incus implant, the smaller end of the implant is placed superiorly in the angle between the upper portion of the malleus and the posterior bony canal wall. The larger end of the implant head rests under the tympanic membrane and is directed inferiorly while the anterior edge of the head lies securely parallel with the malleus handle.

As various changes could be made in the above constructions and methods without departing from the scope of the invention as defined in the claims, it is intended that all matter contained in the above description or shown in the accompanying drawings be interpreted as illustrative and not in a limiting sense.

1. A retaining device for a medical implant, comprising:
(a) an otologic implant, comprising a head and a shaft; and
(b) a sheath, comprising:
(i) an outer surface;
(ii) a bore adapted to receive and retain the shaft of the implant; and
(iii) at least one reference mark for cutting the sheath and implant to obtain a desired shaft length;
wherein the sheath is made of a material which is able to be cut;
wherein the implant is retained in the sheath sufficiently securely to allow a surgeon to cut the implant to a desired size by cutting through the sheath and the implant simultaneously, for decreasing the possibility of damage to the implant.

2. The device of claim 1 wherein the head of the implant is adjacent to a first end of the bore and a diameter of the head is less than a height of the sheath.

3. The device of claim 1 wherein a height of the sheath exceeds a diameter of the head of the implant.

4. The device of claim 1 wherein the outer surface of the sheath further comprises at least one flat surface.

5. The device of claim 1 wherein the height of the sheath is sufficient to allow at least a portion of the outer surface of the sheath to contact the shipping package, thereby retaining the sheath in a shipping package sufficiently securely to decrease the possibility of damage to the implant.

6. The device of claim 1 wherein the shipping package is a blue mold.

7. The device of claim 1 wherein the bore extends the entire length of the sheath.

8. The device of claim 1 wherein the sheath is made of silicone.

9. The device of claim 1 wherein the sheath is made of fluroplast.

10. The device of claim 1 wherein the outer surface is rounded.

11. The device of claim 1 wherein the outer surface is square.

12. The device of claim 1 wherein the outer surface is rectangular.

13. The device of claim 1 wherein the outer surface is triangular.

14. A packaged medical implant, comprising:
(a) an otologic implant, comprising a head and a shaft;
(b) a sheath, comprising:
(i) an outer surface;
(ii) a bore adapted to receive and retain the shaft of the implant; and
(iii) at least one reference mark for cutting the sheath and implant to obtain a desired shaft length; and
(c) a shipping package for receiving and retaining the sheath and implant, wherein the height of the sheath is sufficient to allow at least a portion of the outer surface of the sheath to contact the shipping package, thereby retaining the sheath in the shipping package sufficiently securely, for decreasing the possibility of damage of the implant.

15. The device of claim 14 wherein the head of the implant is adjacent to a first end of the bore and a diameter of the head is less than a height of the sheath.

16. The device of claim 14 wherein a height of the sheath exceeds a diameter of the head of the implant.

17. The device of claim 14 wherein the outer surface of the sheath further comprises at least one flat surface.

18. The device of claim 14 wherein the shipping package is a blue mold.

19. The device of claim 14 wherein the bore extends the entire length of the sheath.

20. The device of claim 14 wherein the sheath is made of silicone.

21. The device of claim 14 wherein the sheath is made of fluroplast.

22. The device of claim 14 wherein the outer surface is rounded.

23. The device of claim 14 wherein the outer surface is square.

24. The device of claim 14 wherein the outer surface is rectangular.

25. The device of claim 14 wherein the outer surface is triangular.

26. A method of retaining a medical implant, comprising:
(a) providing an implant, the implant comprising:
(i) a head, and
(ii) a shaft;
(b) providing a sheath, the sheath comprising:
(i) an outer surface;
(ii) a bore adapted to receive and retain the shaft of the implant; and
(iii) at least one reference mark for cutting the sheath and implant to obtain a desired shaft length;
(c) placing the shaft of the implant into the bore of the sheath;
wherein the sheath is made of a material which is able to be cut;
wherein the implant is retained in the sheath sufficiently securely to allow a surgeon to cut the implant to a desired size by cutting through the sheath and the implant simultaneously, for decreasing the possibility of damage to the sheath.

27. The method of claim 26 wherein the outer surface of the sheath further comprises at least one flat surface.

28. The method of claim 26 wherein the bore of the sheath has a diameter approximately equal to the shaft of the implant.

29. The method of claim 26 wherein the bore of the sheath has a diameter sufficient to engage the shaft of the medical implant.

30. The method of claim 26 further including placing the sheath containing the implant in a shipping package, the sheath having a height sufficient to allow at least a portion of the outer surface of the sheath to contact the shipping package, thereby retaining the sheath in the shipping package sufficiently securely to decrease the possibility of damage to the implant.

31. A method of trimming an otologic implant, comprising:
(a) receiving an otologic implant retained in a sheath, the sheath comprising:
(i) an outer surface;
(ii) a bore adapted to receive and retain a shaft of the implant; and
(iii) at least one reference mark for cutting the sheath and implant to obtain a desired shaft length;
(b) placing the sheath on a cutting surface; and
(c) cutting through the sheath and the shaft of the implant to obtain a desired shaft length.

32. The method of claim 31 wherein the cutting further comprises cutting through the sheath at a reference mark.

33. The method of claim 31 wherein the cutting further comprises cutting through the sheath at one of a plurality of reference marks.

34. The method of claim 32 wherein the reference marks are grooves.

35. The method of claim 32 wherein the reference marks are spaced 0.5 mm apart.

36. A method of installing a medical implant, comprising:
(a) receiving an otologic implant retained in a sheath, the sheath comprising:
(i) an outer surface;
(ii) a bore adapted to receive and retain a shaft of the implant; and
(iii) at least one reference mark for cutting the sheath and implant to obtain a desired shaft length;
(b) placing the sheath on a cutting surface;
(c) cutting through the sheath and the shaft of the implant to obtain a desired shaft length;
(d) removing the implant from the sheath;
(e) washing the implant with saline; and
(f) installing the implant in a patient.

37. The method of claim 36 wherein the placing the sheath on a cutting surface further comprises placing a flat surface of the sheath on a cutting surface.

38. The method of claim 36 wherein the cutting further comprises cutting through the sheath at a reference mark.

39. The method of claim 36 wherein the cutting further comprises cutting through the sheath at one of a plurality of reference marks.

40. The method of claim 39 wherein the reference marks are grooves.

41. The method of claim 39 wherein the reference marks are spaced 0.5 mm apart.

42. The method of claim 36 further comprising receiving a sterile medical implant.

43. A method of installing a medical implant, comprising:
(a) receiving an otological implant for total ossicular replacement retained in a sheath, the sheath comprising:
an outer surface;
a bore adapted to receive and retain a shaft of the implant; and
at least one reference mark for cutting the sheath and implant to obtain a desired shaft length;
(b) placing the sheath on a cutting surface;
(c) measuring the length of the implant needed;
(d) cutting simultaneously through the sheath and the shaft of the implant to obtain a desired shaft length;
(e) removing the implant from the sheath;
(f) washing the implant with saline;
(g) placing a head of the implant in direct contact with a drum in an ear of a patient; and
(h) centering the implant on a stapes footplate in the ear of the patient.

44. A method of installing a medical implant, comprising:
(a) receiving an otological implant for malleus incus replacement having a shaft, a head having an anterior edge, a smaller end and a larger end retained in a sheath, the sheath comprising:
(i) an outer surface;
(ii) a bore adapted to receive and retain a shaft of the implant; and
(iii) at least one reference mark for cutting the sheath and implant to obtain a desired shaft length;
(b) placing the sheath on a cutting surface;
(c) measuring the length of the implant needed;
(d) cutting simultaneously through the sheath and the shaft of the implant to obtain a desired shaft length;
(e) removing the implant from the sheath;
(f) washing the implant with saline;
(g) placing the smaller end superiorly in the angle between an upper portion of a malleus and a posterior bony canal wall;
(h) resting the larger end of the head under a tympanic membrane; and
(i) placing the anterior edge of the head of the implant securely parallel with a malleus handle.
